(19) United States
(12) Patent Application Publication
(10) Pub. No.: US 2026/0290759 A1
(43) Pub. Date: Sep. 24, 2026

(54) PLASMA PROBE

(71) Applicant: Erbe Elektromedizin GmbH, Tuebingen (DE)

(72) Inventors: Uwe SCHNITZLER, Tuebingen (DE); Daniel SADLER, Tuebingen (DE)

(21) Appl. No.: 19/560,542

(22) Filed: Mar. 9, 2026

(30) Foreign Application Priority Data
Mar. 18, 2025 (EP) 25164477.9

(51) Int. Cl. H01J 37/32

(52) U.S. Cl. CPC H01J 37/32541; H01J 37/3244; H01J 37/32568; H01J 37/32577; H01J 2237/336

(57) ABSTRACT

A plasma probe for coagulation and/or ablation of biological tissue of a patient is disclosed. It includes a hose that extends from a proximal hose end to a distal hose end and a probe head arranged on the distal hose end. The probe head comprises an electrode cage formed from multiple electrodes and extending from a proximal head end to a distal head end. The electrodes laterally project beyond the outer circumference of the hose at least in a section. The probe head additionally includes a central element and a centering element, wherein the central element defines a center axis of the probe head. The centering element is arranged at the distal head end. The electrodes of electrode cage are movable along the center axis relative to the central element.

### CROSS REFERENCE TO RELATED APPLICATION

This application claims the benefit of European Patent Application No. 25164477.9, filed Mar. 18, 2025, which is incorporated herein by reference in its entirety.

### TECHNICAL FIELD

The invention refers to a plasma probe for (large-scale) coagulation or ablation of biological tissue of a human or animal patient.

### BACKGROUND

Plasma probes, such as plasma coagulation probes or plasma ablation probes, are generally known. They are medical instruments that are used in the electro-surgery in order to coagulate or remove biological tissue by means of application of plasma. The coagulation medically serves to seal or obliterate tissue. Contrary to this the ablation medically serves to specifically remove or destroy tissue. Particularly in specific endoscopic uses a treatment of the tissue with the largest area possible is advantageous in order to reduce the duration of the medical surgery.

A catheter device for ablation of mucosa by means of argon plasma is described in WO 2019/089392 A1. The catheter device comprises one or more electrodes as well as a spreadable cage structure, which can consist of hoses or a net and through which a uniform outlet of the treatment gas is facilitated. In another embodiment the gas flow of the treatment gas is uniformly distributed by means of a double wall balloon.

EP 4 282 363 A2 describes a device for tissue ablation having spreadable cage shaped electrodes. The cage-shaped electrodes can be movable and/or retractable as well as radially spreadable. The radially extendable electrodes can comprise one or more balloons, a cage and radially extendable arms.

DE 103 50 709 A1 moreover describes an instrument for coagulation of tissue that is configured for supply of argon gas in a space between an electrode inside the probe and the tissue. Thereby, a guide device for deflection of the gas flow in a predefined direction is described.

Further, EP 3 769 707 A1 describes a medical instrument in which the electrode is provided with a heat dissipation device, so that the heat resistance of the electrode measured in longitudinal direction can be increased.

### SUMMARY

Starting therefrom it is one object of the invention to provide an improved plasma probe by means of which particularly a larger treatment surface can be obtained, that shall allow a position-independent ignition of the plasma.

The plasma probe according to the invention is configured for coagulation of biological tissue of a patient.

The plasma probe comprises a hose that extends from a proximal hose end to a distal hose end and comprises a probe head arranged on the distal probe end.

The hose head of the plasma probe comprises an electrode cage, at least one gas outlet, a centric central element and a centering element.

The electrode cage is formed of multiple electrodes and extends from a proximal head end to a distal head end. Preferably, the electrodes are arranged in a preferred position, so that at least a section of the electrode cage projects laterally beyond the outer circumference of the hose. The preferred position of the electrodes of the electrode cage thereby particularly is the spatial position and/or orientation in which the electrodes come to rest on their own without application of external forces. In this preferred position the internal tensions and/or deformations of the electrodes are minimum, so that the electrodes steadily rest in this preferred position without expenditure of force.

For example, the electrode cage can comprise 4, 5, 6 or more electrodes. The electrodes can consist of metal wire, for example of stainless steel, platinum, titanium, gold, silver, tungsten or graphite or carbon fiber material. The electrodes of the electrode cage are preferably electrically and preferably thermically connected with an electrical supply line at the distal hose end. The electrical supply line extends inside the hose from the distal hose end to the proximal hose end, where the hose is configured to connect the electrical supply line to a supply device, for example by means of a specific connector.

The gas outlet is arranged at the proximal head end and can be supplied at the proximal hose end with the gas that shall be output. The hose can comprise at least one gas channel that extends from the distal hose end up to the proximal hose end, wherein the hose is configured for connection of the gas channel with the supply device, for example via a specific connector. For example, the gas can be argon, helium, nitrogen or oxygen.

The centric central element defines a center axis of the probe head. The centering element is arranged at the distal end of the probe head, wherein the electrodes of the electrode cage are connected with the centering element and can be moved along the center axis relative to the central element.

The centering element can be axially (along the center axis) movably arranged relative to the central element. The central element can form a guide for the centering element. Particularly the electrodes are axially immovably (rigidly) fixated at the proximal head end. The centering element can also be axially immovable if the electrodes are movable along the center axis relative to the centering element.

The centering element can be rotationally symmetrically configured relative to the center axis in disc-shaped manner. Preferably the centering element comprises a passage opening. The passage opening can be dimensioned so that the central element extends therethrough along the center axis. Preferably the centering element has connection positions at its circumference, where the electrodes of the electrode cage are connected to the centering element. Preferably the electrodes are connected with the centering element in a friction-fit and/or form-fit manner. The centering element and the electrode cage can also be monolithically configured. The central element can form a guide for the centering element. Particularly, the electrodes are axially immovably (rigidly) fixated at the proximal head end. The centering element can be rotationally symmetrically configured relative to the center axis in disc-shaped manner. Preferably the centering element comprises a passage opening. The passage opening can be dimensioned, so that the central element extends therethrough along the center axis. Particularly the centering element can be monolithically configured. Preferably the centering element comprises connection positions at its circumference, where the electrodes can be connected to the centering device.

The central element extends particularly from the proximal head end to the distal head end of the plasma probe. Preferably, the central element is configured as flushing nozzle that is configured to output a flushing fluid at the distal head end, particularly a flushing liquid. The central element can be at least substantially cylindrically configured. In the central element a channel can be formed extending axially along the center axis. The channel can open out into a flushing opening, which is formed on the distal head end of the central element. At the proximal head end the channel can be fluidically connected with a flushing fluid supply line. The flushing fluid supply line can extend from the distal hose end up to the proximal hose end. The flushing fluid supply line is particularly arranged in a manner extending centrally inside the hose. The flushing fluid supply line can be a flexible hose, such as a capillary, for example.

The electrodes of the electrode cage can spring elastically deform because of the moveable centering element at the distal head end axially. In doing so the electrodes can be changed elastically with regard to the diameter and the axial position independent from the central element. When inserting the plasma probe into the body of the patient, for example through an endoscope, or during retracting the plasma probe, forces can be applied onto the electrode body externally. Due to the elastic resilience of the electrodes, supported by the movability of the centering element relative to the central element, the electrodes can be moved out of the preferred position, whereby the resistance for the user during insertion or retraction of the plasma probe can be reduced. Moreover, the risk is reduced that the electrodes of the electrode cage are permanently bent in a manner making the application surface smaller compared to the case where the electrodes are arranged in the preferred position.

Particularly the electrode cage comprises an envelope that is rotationally symmetrical around the center axis. Preferably the electrodes comprise positions that are furthest distanced from the center axis in the preferred position. These positions are particularly arranged inside a section whose distance from the center axis exceeds the outer circumference of the hose.

The electrode cage can be domed, particularly pear-shaped. Particularly, the electrode cage comprises a first sub-section adjoining the distal head end and a second sub-section adjoining the proximal head end.

The first sub-section and the second sub-section of the electrode cage can be symmetrically or asymmetrically configured in relation with each other. Preferably, the first sub-section of the electrode cage has a larger curvature than the second sub-section of the electrode cage, so that the first sub-section can be configured more bulbous than the second sub-section. This geometric configuration of the electrode cage supports the ignition ability of the plasma probe, even in case of particularly acute angles between the tissue surface of the patient and the probe head of the plasma probe, particularly between the tissue surface of the patient and the center axis of the probe head. Due to the lower curvature the second sub-section is configured in a more oblong manner than the first sub-section, whereby the retraction of the probe head from the endoscope can be simplified. The electrodes can spring radially inwardly.

It is preferred that the electrode cage is asymmetric relative to a plane extending radial to the center axis and through the position having the largest outer circumference of the electrode cage being in the preferred position. The position having the largest outer circumference is particularly closer to the distal head end than to the proximal head end. This position can be defined by points on the electrodes that have the greatest distance to the center axis along the extension of the electrodes from the proximal to the distal head end. Particularly the electrode cage is asymmetric or non-mirror symmetric relative to this plane.

Preferably, the centering element consists of a material that comprises a thermal conductivity that is equal or larger than the thermal conductivity of the electrodes of the electrode cage, so that the centering element can absorb and dissipate the heat produced during the ignition process. The plasma probe usually applies in contactless manner, however in case of a large-scale application zone, that means coagulation and/or ablation zone, as it is provided in the case of a large-scale electrode cage, it cannot be excluded that an unintentional contact between the electrodes and the tissue occurs. The dissipation of the heat allows to avoid sticking of tissue portions and coagulated liquids in case of contact between the electrode cage or the centering element and the tissue, whereby the visibility and the safe function of the medical instrument can be maintained. Additionally, due to the increased thermal conductivity of the centering element, the heat produced during the ignition process can be absorbed and dissipated. The danger of a thermal damage of the distal components, such as electrodes, centering element and central element, which is present due to the high thermal stress during the ignition process and the plasma application, can thus be reduced.

Particularly, an electrical supply line is arranged inside the hose that is connected with the electrodes of the electrode cage at the distal hose end. The electrical supply line preferably consists of a material that comprises a higher thermal conductivity than the electrodes of the electrode cage. This allows to absorb the heat created during the ignition process via the electrical supply line and to conduct or distribute it away from the probe head in direction toward the proximal hose end. The electrical supply line can consist of carbon fibers, for example.

Alternatively or additionally, the electrical supply line can be provided with a coating whose material comprises a higher thermal conductivity than the electrodes of the electrode cage. In doing so, the thermal conductivity of the electrical supply line can be improved.

Thereby, the electrical supply line can absorb the heat created during the ignition process and can dissipate it away from the probe head in direction toward the proximal hose end. For example, the electrical supply line can be a wire having a silver coating or a copper wire having a polyamide coating.

Gas can exit from the gas outlet arranged at the proximal head end in direction toward the distal head end. The central element can be configured as gas guiding device. Preferably, the central element is configured to guide the gas exiting the gas outlet laterally (transverse to the center axis) in direction toward the electrodes of the electrode cage. Particularly the contour of the central element is such that the gas exiting the gas outlet is deflected transverse to the center axis (laterally outward). This can result in an improvement of the gas distribution at the probe head, whereby particularly an improved lateral ignition ability can be achieved, whereby the gas surrounds the electrodes of the electrode body. Transverse to the center axis means particularly orthogonal to the center axis.

Preferably, the radius of the central element varies along the center axis, so that the gas flows laterally outwardly. The radius of the central element can taper (continuously) from the distal head end to the proximal head end. The central element can comprise at least one guide lip. The guide lip can be particularly arranged in an exit area of the gas outlet, so that the gas flow exiting the gas outlet impacts on the guide lip. The guide lip can particularly create turbulences in the gas flow, whereby a better distribution of the exiting gas can be achieved, particularly in lateral direction. The guide lip can be configured to guide the gas flow transverse to the center axis laterally outwardly.

The central element can be connected with a flushing fluid supply line at the proximal head end. The flushing fluid supply line can be guided out of the hose at the proximal head end. The central element and the flushing fluid supply line can then be connected with each other outside the hose in the area of the probe head.

Particularly, the electrodes are arranged at the proximal head end in a ring gap forming the gas outlet between the flushing fluid supply line and the hose. Alternatively, the electrodes can be arranged at the proximal head end in a ring gap forming the gas outlet between the central element and the hose.

Preferably the electrodes can be arranged, so that they interrupt the ring gap, whereby the gas can exit from multiple ring segments between the electrodes in direction toward the distal head end.

Preferably, the electrodes are immovable on the proximal head end of the probe head, that is immovably connected in direction toward the center axis. For example, the electrodes can be immovably attached at the proximal head end by means of a clamping connection. For example, the electrodes have a larger diameter than the gap width of the ring gap. This oversize can result in that the electrodes are clamped between the central element and the hose or between the flushing fluid supply line and the hose. In this form the electrodes of the electrode cage are immovable at the proximal head end and movable relative to the central element at the distal head end.

### BRIEF DESCRIPTION OF THE DRAWINGS

Further details, advantageous embodiments of the invention are derived from the dependent claims, the drawings and the description. The drawings show:

FIG. 1 an example of the plasma probe according to the invention with a supply device to which the plasma probe is connected, in a perspective illustration;

FIG. 2 an example of the probe head of the plasma probe in a perspective illustration;

FIG. 3 a cross-section of the hose of the plasma probe at the distal hose end;

FIG. 4 a longitudinal cut through the probe head of the plasma probe;

FIGS. 5 to 8 additional embodiments of the probe head of the plasma probe in perspective illustration.

### DETAILED DESCRIPTION

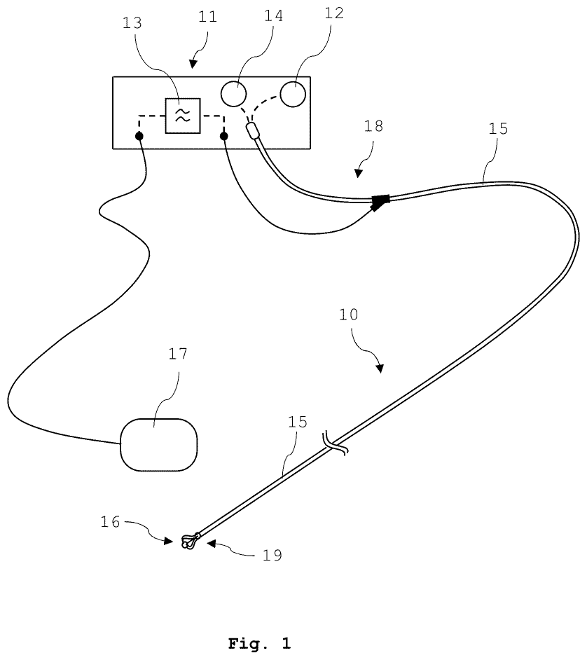

FIG. 1 shows an example of a plasma probe 10 according to the invention that is configured for coagulation of biological tissue of a patient, and a supply device 11 to which the plasma probe 10 is connected. The invention is explained in FIG. 1 based on an argon plasma probe that is configured for coagulation of biological tissue. However, the concept according to the invention can also be used for any other plasma probe.

The supply device 11 comprises a gas source 12, a generator 13 for electrical supply of the plasma probe 10 and a flushing fluid source 14 for supply of the plasma probe 10 with a flushing fluid. The gas source 12 in FIG. 1 is an argon gas source.

The plasma probe 10 comprises a hose 15 and a probe head 16. The hose 15 is connected with the supply device 11 via respective connection means, so that the probe head 16 can be supplied with all required media via hose 15. The plasma probe 10 is connected to the supply device 11 at a proximal end 18. The hose 15 of plasma probe 10 extends from the proximal hose end 18 up to a distal hose end 19 on which probe head 16 of plasma probe 10 is arranged.

The generator 13 creates electrical, high-frequency (HF) currents that are supplied to the probe head 16 via an electrical supply line extending in the hose 15. The generator 13 is additionally connected with a neutral electrode 17 via respective connection means, wherein the neutral electrode 17 is to be attached to the patient prior to the use of the plasma probe.

The following description, however, applies also for plasma probes with other configurations of the neutral electrode.

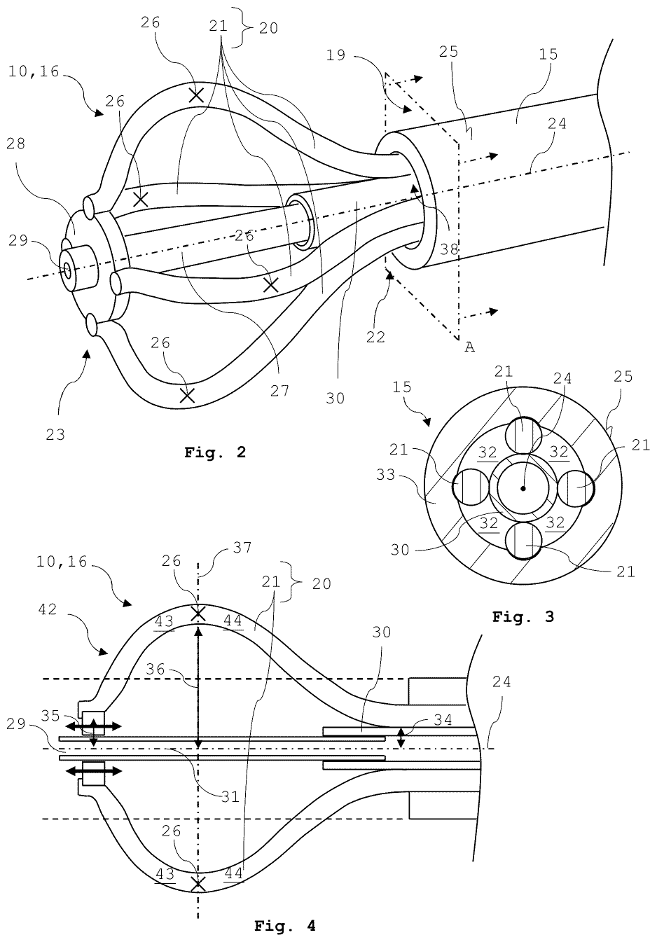

FIG. 2 shows an example of a probe head 16 of the plasma probe 10 in a perspective view. The probe head 16 comprises an electrode cage 20 formed of multiple electrodes 21. In the example shown in FIG. 2 the electrode cage 20 consists of four electrodes 21. Different to the illustration in FIG. 2, the electrode cage 20 can also be configured of more than four electrodes 21. The electrodes 21 of electrode cage 20 extend from a proximal head end 22 to a distal head end 23 of probe head 16.

In the center of electrode cage 20 a center axis 24 is formed that particularly extends from the proximal head end 22 to the distal head end 23. Centrically relative to electrode cage 20, the probe head 16 comprises a central element 27 extending along the center axis 24 from the proximal head end 22 toward the distal head end 23.

The electrodes 21 of electrode cage 20 laterally project transverse to the center axis 24 beyond an outer circumference 25 of hose 15, at least partly. The individual electrodes 21 are particularly convex. Between the proximal head end 22 and the distal head end 23 they have positions 26 where the outer circumference of the electrodes 21 in lateral direction is largest. At the proximal head end 22 and the distal head end 23 the outer circumference of the electrode 21 is smaller than at the positions 26.

At the distal head end 23 probe head 16 additionally comprises a centering element 28 to which the individual electrodes 21 of electrode cage 20 are attached. In the example illustrated in FIG. 2 the electrodes 21 are immovably attached to the centering element. For example, the electrodes 21 can be rigidly clamped to the centering element 28. The connection can also be a substance bond connection, for example a solder joint, an adhesive joint or another comparable connection. If the electrodes 21 are immovably connected with the centering element, the centering element 28 can be moved along the center axis 24 relative to the central element 27.

Additionally or alternatively the electrodes 21 can be movably connected relative to the centering element 28 along the center axis 24 with the centering element 28. The centering element 28 can then be immovably connected with the central element 27. The centering element 28 and the central element 27 can be configured monolithically if the electrodes 21 are movable relative to the centering element 28 along the center axis 24.

In FIG. 2 centering element 28 is arranged in a manner seated on the central element 28 and can be moved relative to the central element 27. The individual electrodes 21 of electrode cage 20 are in this manner attached on the distal head end 23 at fixed positions relative to one another. However, the ends of the electrodes 21 are movable by means of the centering element 28 along the center axis 24 relative to the central element 27.

The central element 27 depicted in FIG. 2 is configured as flushing nozzle through which a flushing fluid, such as water, can be output in distal direction from the probe head 16.

The central element 27 comprises for this purpose a flushing opening 29 arranged at the distal end 23 of probe head 16.

A channel 31 can be configured inside central element 27 (as shown in FIG. 4), which extends along the center axis 24 and opens out into flushing opening 29. The channel 31 is fluidically connected with a flushing fluid supply line 30 at the proximal head end 22 of probe head 16. The flushing fluid supply line 30 extends from the distal hose end 19 up to the proximal hose end 18.

The flushing fluid supply line 30 can be a small tube, such as a thin, flexible plastic hose, for example a capillary tube. The flushing fluid supply line 30 extends particularly centrically from the distal hose end 19 inside hose 15 up to the proximal hose end 18, where it can be connected to a flushing fluid source 14 of supply device 11.

In FIG. 3 a cross-section of hose 15 along cutting plane A at the distal hose end 19 is shown.

The flushing fluid supply line 30 is centrally arranged inside hose 15. The electrodes 21 of electrode cage 20 are arranged adjacent to the wall of the flushing fluid supply line 30.

The electrodes 21 are uniformly arranged distributed along the outer circumference of the flushing fluid supply line 30 in a ring gap. The ring gap can extend from the proximal hose end 18 up to the distal hose end 19.

At least at the distal hose end 19 the electrodes 21 divide the ring gap in gap segments 32 forming the gas outlet 38 at the proximal head end 22 of plasma probe 10. Through the gap segments 32 of gas outlet 38 argon gas exits from hose 15 at the proximal head end 22 of probe head 16 in direction toward the distal head end 23.

The electrodes 21 can at least partly extend further into hose 15, as depicted in FIG. 3. Inside hose 15 an electrical supply line is arranged that is connected with the electrodes 21 of electrode cage 20 at the distal hose end 19.

The electrical supply line can be formed by one or more individual wires, mesh wires, from carbon fibers or also by a metallized surface, for example on the outer circumference of flushing fluid supply line 30 or on the inner circumference of hose sheath 33.

Around the individual electrodes 21 a hose sheath 33 is arranged. The electrodes 21 are particularly provided with a certain oversize so that the electrodes 21 are clamped between tube 30 and hose sheath 33. The clamping connection results particularly in that the ends of electrodes 21 are immovably arranged at the proximal head end 22 in axial direction along the center axis 24.

FIG. 4 illustrates a longitudinal cut through probe head 16 of plasma probe 10. The centering element 28 is movably arranged in axial direction on central element 27. In doing so, the electrodes 21 of electrode cage 20 can dodge at the distal head end 23 of probe head 16 if external forces act on the electrodes. Additionally, the central element 27 configured as a nozzle can be moved independent from the electrodes 21.

The electrodes 21 extend from the proximal head end 22 to the distal head end 23, whereby a section 42 of the electrodes 21 projects laterally relative to the center axis 24 beyond the outer circumference 25 of hose 15. The section 42 extending beyond the outer circumference 25 of hose 15 is preferably longer (in axial direction) than the remaining sections of the electrode cage 20.

The distance 34 between the electrodes 21 and the center axis 24 at the proximal head end 22 is shorter than the distance 35 of electrodes 21 to the center axis 24 at the distal head end 23. At a position 26 the distance 36 between electrode 21 and center axis 24 is longest.

The electrode cage 20 comprises a first sub-section 43 and a second sub-section 44.

Both sub-sections 43, 44 adjoin the position 26 having the largest outer circumference. The first sub-section 43 adjoins the distal head end 23 and the second sub-section adjoins the proximal head end 22.

In FIG. 4 the first sub-section 43 comprises a greater curvature than second sub-section 44. The position 26 is particularly not centered between the proximal head end 22 and the distal head end 23. Preferably, the position 26 is closer to the distal head end 23 than to the proximal head end 22.

The electrodes 21 are not mirror symmetrically relative to a plane 37 extending through the position 26. The electrode cage 20 is bulbously configured in direction toward the distal head end 23. This improves the ignition ability of plasma probe 10, particularly in case of comparably flat angles between probe head 16 and the tissue.

Gas exits from the gas outlet 38, which is covered by electrodes 21 in FIG. 4, at the proximal head end 22 in direction toward the distal head end 23. The centering element 28 can result in a turbulence of the gas flow, so that the gas can accumulate proximally in front of the centering element 28 and can be distributed laterally transverse to the center axis 24. This can be additionally advantageous for the ignition behavior of plasma probe 10.

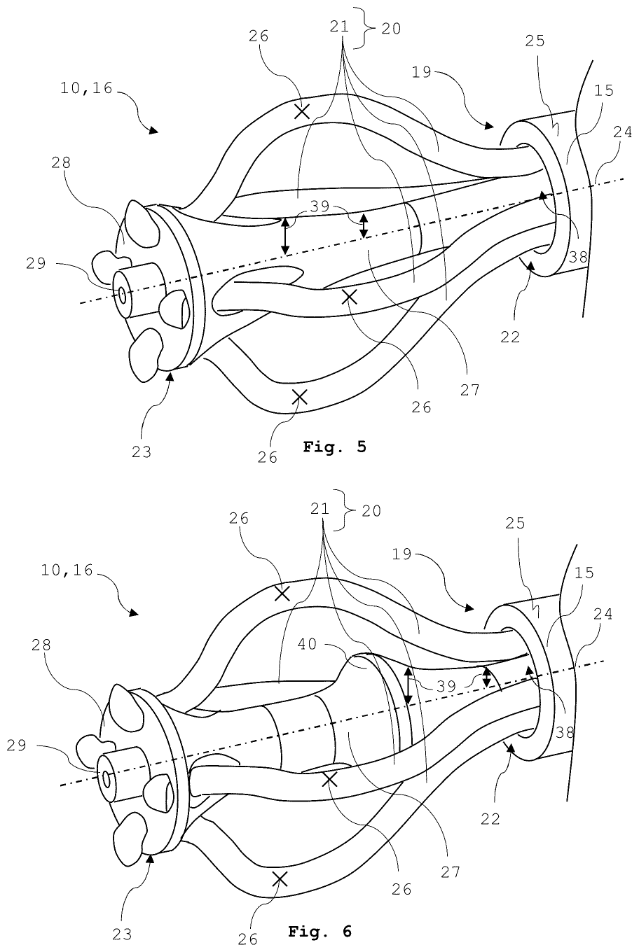

FIG. 5 shows another example of the probe head 16 of the plasma probe 10. For the example shown in FIG. 5, the above explanations regarding the reference symbols apply accordingly.

The example shown in FIG. 5 distinguishes from the example shown in FIG. 2 in that the central element 27 is configured to guide the gas flow exiting at the proximal head end 22 transverse to the center axis 24 laterally outwardly.

The radius 39 of central element 27 varies from the proximal head end 22 toward the distal head end 23. In this example the radius 39 increases continuously.

FIG. 6 shows another example in which in modification to the preceding example the central element 27 additionally comprises a guide lip 40 that is configured to guide the gas flow laterally outwardly. The guide lip 40 is arranged between the position 26 and the proximal head end 22, so that the gas flow 38 is deflected laterally in direction toward the position 26. Additionally, turbulences in the exiting gas flow can be produced by means of guide lip 40, which can result in a more uniform gas distribution in the area of the electrode cage 20.

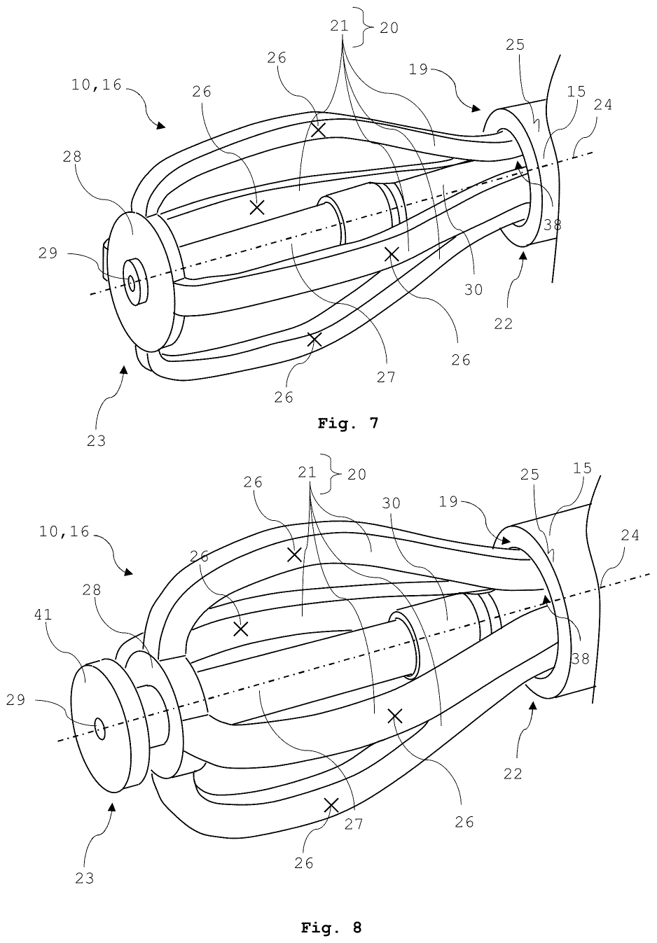

FIG. 7 illustrates another example of the probe head in which the electrodes 21 of electrode cage 20 as well as the centering element 28 consist of a bent sheet metal part. The centering element 28 and the electrodes 21 of electrode cage 20 are monolithically configured.

FIG. 8 shows another example of the probe head of plasma probe 10. In the example shown in FIG. 8 the electrodes 21 of electrode cage 20 are monolithically configured with centering element 28. In this embodiment central element 27 comprises a stop 41 on the distal head end 23. The stop 41 is configured as projection that projects transverse to the center axis 24 from central element 27 from the surface of central element 27 distal relative to the centering element 28. The stop 41 limits the movement stroke of centering element 28, so that the centering element cannot distally slide from central element 27 serving as guide. The electrodes 21 and the centering element consist of carbon fibers in this example.

The plasma probe 10 described so far operates as follows:

The plasma probe 10 is connected with the supply device 11, that means the flushing fluid source 14, the HF generator 13 and the gas source 12 are connected with the proximal hose end 18 of hose 15.

For example, plasma probe 10 can be inserted into the body interior of the patient, for example in the stomach of the patient.

During such applications, particularly a large stomach surface shall be treated. For the ablation of the stomach surface the gas introduced at the proximal hose end 18 is conveyed to the distal hose end 19 to which the probe head 16 of plasma probe 10 is attached. At the probe head 16 the argon gas flow exits.

Concurrently the electrodes 21 of electrode cage 20 are supplied with HF currents from generator 13 via line 14. The argon gas can be laterally guided to the electrodes 21, for example by means of the intermediate element 27 configured as guide body, so that a large-scale and angle-independent ignition and plasma creation are possible.

After the application the plasma probe can be retracted via the endoscope from the stomach of the patient. During insertion and retraction of plasma probe 10 the electrodes 21 of electrode cage 20 can deflect elastically.

The plasma probe 10 according to the invention serves for coagulation and/or ablation of biological tissue of a patient. It comprises a hose 15 that extends from a proximal hose end 18 to a distal hose end 19 and a probe head 16 arranged on the distal hose end 19. The probe head 16 comprises an electrode cage 20 formed from multiple electrodes 21 and extending from a proximal head end 22 to a distal head end 23. The electrodes 21 take a preferred position in which they laterally project beyond the outer circumference 25 of hose 15 at least in a section 42. The probe head 16 additionally comprises a central element 27 and a centering element 28, wherein the central element 27 defines a center axis 24 of probe head 16. At the distal head end 23 centering element 28 is arranged. The electrodes 21 of electrode cage 20 are movable along the center axis 24 relative to the central element 27.

### REFERENCE SIGNS

10 plasma probe
11 supply device
12 gas source
13 generator
14 flushing fluid source
15 hose
16 probe head
17 neutral electrode
18 proximal hose end
19 distal hose end
20 electrode cage
21 electrodes of electrode cage
22 proximal head end
23 distal head end
24 center axis
25 outer circumference of hose
26 position having maximum outer circumference
27 center element
28 centering element
29 flushing opening
30 flushing fluid supply line (tube, capillary tube)
31 channel inside center element
32 gap segment
33 hose sheath
34 distance to center axis at proximal head end
35 distance to center axis at distal head end
36 distance to center axis at position having maximum circumference
37 plane
38 gas outlet
39 radius of center element
40 guide lip
41 stop
42 section
43 first sub-section
44 second sub-section
A cutting plane

1. A plasma probe (10) configured for coagulation and/or ablation of biological tissue of a patient, the plasma probe (10) comprising:
a hose (15) extending from a proximal hose end (18) to a distal hose end (19);
a probe head (16) at the distal hose end (19), wherein the probe head (16) comprises:
an electrode cage (20) comprising multiple electrodes (21) that extend from a proximal head end (22) to a distal head end (23);
at least one gas outlet (38) at the proximal head end (22) for emitting a gas supplied via the hose (15);
a central element (27) that defines a center axis (24) of the probe head (16); and
a centering element (28) at the distal head end (23), wherein the multiple electrodes (21) of the electrode cage (20) are connected with the centering element (28) and are movable relative to the central element (27) along the center axis (24).

2. The plasma probe (10) according to claim 1, wherein at least a portion of the multiple electrodes (21) of electrode cage (20) project laterally beyond an outer circumference (25) of the hose (15).

3. The plasma probe (10) according to claim 1, wherein the electrode cage (20) comprises a first sub-section (43) and a second sub-section adjoining the first sub-section (43) axially, wherein the first sub-section (43) adjoins the distal head end (23) and the second sub-section (44) adjoins the proximal head end (22).

4. The plasma probe (10) according to claim 1, wherein the electrode cage (20) is asymmetric relative to a plane (37) extending radially relative to the center axis (24) through a position where the electrode cage (20) has a largest outer circumference.

5. The plasma probe (10) according to claim 1, wherein the centering element (28) comprises a material having a thermal conductivity that is equal to or higher than a thermal conductivity of the multiple electrodes (21) of the electrode cage (20).

6. The plasma probe (10) according to claim 1, further comprising an electrical supply line arranged in the hose (15) that is connected with the multiple electrodes (21) of the electrode cage (20) at the distal hose end (19), wherein the electrical supply line comprises a material having a thermal conductivity that is equal to or higher than a thermal conductivity of the multiple electrodes (21) of the electrode cage (20).

7. The plasma probe (10) according to claim 6, wherein the electrical supply line comprises a coating having a higher thermal conductivity than a thermal conductivity of at least one of a core material of the electrical supply line and a material of the multiple electrodes (21) of the electrode cage (20).

8. The plasma probe according to claim 1, wherein the central element (27) guides the gas exiting the at least one gas outlet transversely relative to the center axis

9. The plasma probe (10) according to claim 1, wherein an outer circumference of the central element (27) increases from the proximal head end (22) toward the distal head end (23).

10. The plasma probe according to claim 1, wherein the central element (27) comprises a guide lip (40) that projects laterally beyond an adjacent outer circumferential surface of the central element (27).

11. The plasma probe (10) according to claim 1, wherein the multiple electrodes (21) are arranged in a ring gap at the proximal head end (22).

12. The plasma probe (10) according to claim 1, wherein the multiple electrodes are immovably attached to the probe head (16) at the proximal head end (22).

13. The plasma probe (10) according to claim 1, wherein the central element (27) comprises a flushing nozzle that is outputs a flushing fluid at the distal head end (23).

14. The plasma probe (10) according to claim 1, further comprising a flushing opening (29) that outputs a flushing fluid is formed in the central element (27) at the distal head end (23).

15. The plasma probe (10) according to claim 14, further comprising a channel (31) in the central element (27) that extends along the center axis (24) from the proximal head end (22) to the flushing opening (29) at the distal head end (23).

16. The plasma probe according to claim 15, wherein the channel (31) is fluidically connected with a flushing fluid supply line (30) at the proximal head end (22) and extends from the proximal hose end (18) up to the distal hose end (19).
